(19) United States
(12) Patent Application Publication
(10) Pub. No.: US 2003/0208784 A1
(43) Pub. Date: Nov. 6, 2003

(54) Methods of constructing a gene mutation library and compounds and compositions thereof

(76) Inventors: Henry Earl Ruley, Nashville, TN (US); Geoffrey G. Hicks, Nashville, TN (US)

Correspondence Address: NEEDLE & ROSENBERG, P.C., SUITE 1000, 999 PEACHTREE STREET, ATLANTA, GA, 30309-3915, US

(21) Appl. No.: 10/454,974

(22) Filed: Jun. 4, 2003

Related U.S. Application Data
(63) Continuation of application No. 09/039,161, filed on Mar. 13, 1998, now abandoned.
(60) Provisional application No. 60/040,538, filed on Mar. 13, 1997.

(51) Int. Cl. A01K 67/00; C12Q 1/68; C07H 21/04; C12N 5/08

(52) U.S. Cl. 800/13; 435/6; 435/366; 536/23.2

(57) ABSTRACT

The invention is directed toward a method of producing a selected cell line or a non-human transgenic animal model for the analysis of the function of a gene comprising introducing into an embryonic stem cell a vector having a selectable marker which, when the vector is inserted within a gene, the inserted vector can inhibit the expression of the gene, selecting embryonic stem cells expressing the selectable marker, excising the vector from the embryonic stem cells expressing the selectable marker such that host DNA from the gene is linked to the excised vector, sequencing the host DNA in the excised vector, comparing the sequence of the host DNA to known gene sequences to determine which host DNA is from a gene for which a model for the analysis of the function the gene is desired, selecting the embryonic stem cell containing the inhibited gene for which a model for the analysis of gene function is desired, and forming a cell line or a non-human transgenic animal from the selected embryonic stem cell. The invention is also directed toward a method of selecting a cell for the analysis of the function of a gene. The invention is also directed toward libraries of cells, cell lines, and transgenic animals produced using cells produced by the methods disclosed herein.

This application claims priority to U.S. Provisional Application Serial No. 60/040,538, which was filed on Mar. 13, 1997, the contents of which are incorporated herein.

This invention was made with government support under RO1 HG00684 awarded by the National Institutes of Health. The government has certain rights in the invention.

### BACKGROUND OF THE INVENTION

1. Field of the Invention

This invention relates generally to methods of producing or selecting cells or transgenic animals containing inhibited genes for the analysis of gene function.

2. Background Art

The molecular analysis of mammalian genomes is expected to provide insights concerning gene function and will assist efforts to identify genes important in human disease. Genetic approaches, successful in lower organisms, are unsuited for mammals given the size of their genomes, long reproduction cycles, and costs of housing animals. Physical methods have therefore dominated efforts to study mammalian gene functions and have reached the point that large-scale genome sequencing is now a feasible undertaking. The sequence of the S. cerevisiae genome is already complete, Drosophila and C. elegans genome sequences are progressing rapidly, and most human genes will be characterized in the next few years by assembling expressed sequence tags (ESTs) into larger contiguous transcripts (1).

While impressive, the expanding wealth of sequence information greatly outpaces our understanding of gene functions. Nearly 50% of yeast genes and non-redundant mammalian ESTs are unrelated to the known genes of any organism, and sequence similarities do not necessarily predict biological function (2). Moreover, relatively few spontaneous mutations in mammalian genes are available for study, and most of these involve dominant post-natal phenotypes (3-5).

A functional analysis of most mammalian genes within the context of the organism will therefore require new methods to study gene functions in vivo. Particularly important in this regard, has been the use of embryonic stem (ES) cell lines to construct mouse strains in which gene functions have been mutationally inactivated (6). In principle, it is possible to construct embryonic stem cells with mutations in any cloned gene. However, for genes cloned initially as cDNAs, one must isolate-and characterize genomic clones, construct targeting vectors, screen ES clones to identify those in which the genes have been disrupted, and develop cell lines or chimeric non-human cells capable of passing the mutant gene into the germline. While over 700 genes have been disrupted in this manner (7), the process is too slow and labor intensive for large scale mutagenesis.

To address this problem, gene trapping strategies have been developed to disrupt genes expressed in mouse embryonic stem cells (8-14). A promoter-less selectable marker is introduced into cells, either by transfection or by retrovirus transduction, and clones expressing the marker gene are selected when the targeting vector inserts into, and disrupts, expressed cellular genes. Large numbers of mutant clones can be analyzed for significant mutations, including those that give rise to mutant phenotypes following germline transmission, that target developmentally regulated genes (9, 11, 13, 15, 16), that disrupt genes regulated by extracellular agonists (17), or that affect genes encoding secreted and transmembrane proteins (12).

Screens involving the phenotypic analysis of ES cells and mice are still too slow and expensive for large-scale mutagenesis. Moreover, genes associated with interesting phenotypes or lacZ expression patterns must be cloned and characterized on a case-by-case basis. While this may lead to the discovery of new genes, the process still requires some effort, and in the end, the mutations may affect previously characterized genes or gene sequences (13). Moreover, the task of gene discovery will be largely accomplished as a result of large scale cDNA sequencing efforts. Thus, within the next few years, the vast majority of inserts will disrupt characterized gene sequences.

The present invention therefore provides a valuable and widely needed method of a sequence-based screen to identify cellular genes disrupted as a result of provirus integration. The process (designated “tagged sequence mutagenesis”) involves sequencing a short segment of DNA from each targeted gene and using the sequences to search the nucleic acid databases. Sequence-based screens are be faster and less expensive than screens based on cellular or organismal phenotypes. Large numbers of ES cell clones can be analyzed and cryopreserved, providing a library of sequenced mutations available for transmission into non-human germline cells. Finally, the sequence tags provide highly portable information about each mutation. Once they have been entered into the nucleic acid databases, any investigator can learn of mutations in a specific gene of interest simply by searching the database with the appropriate gene sequence.

### SUMMARY OF THE INVENTION

In accordance with the purpose(s) of this invention, as embodied and broadly described herein, this invention, in one aspect, provides a method of producing a selected cell line or a non-human transgenic animal model for the analysis of the function of a gene comprising introducing into an embryonic stem cell a vector having a selectable marker which, when the vector is inserted within a gene, the inserted vector can inhibit the expression of the gene, selecting embryonic stem cells expressing the selectable marker, excising the vector from the embryonic stem cells expressing the selectable marker such that host DNA from the gene is linked to the excised vector, sequencing the host DNA in the excised vector, comparing the sequence of the host DNA to known gene sequences to determine which host DNA is from a gene for which a model for the analysis of the function the gene is desired, selecting the embryonic stem cell containing the inhibited gene for which a model for the analysis of gene function is desired, and forming a cell line or a non-human transgenic animal from the selected embryonic stem cell.

The invention further provides a library of embryonic stem cells and non-human transgenic animals produced by selecting a cell line or a non-human transgenic animal model for the analysis of the function of a gene comprising introducing into an embryonic stem cell a vector having a selectable marker which, when the vector is inserted within a gene, the inserted vector can inhibit the expression of the gene, selecting embryonic stem cells expressing the selectable marker, excising the vector from the embryonic stem cells expressing the selectable marker such that host DNA from the gene is linked to the excised vector, sequencing the host DNA in the excised vector, comparing the sequence of the host DNA to known gene sequences to determine which host DNA is from a gene for which a model for the analysis of the function the gene is desired, selecting the embryonic stem cell containing the inhibited gene for which a model for the analysis of gene function is desired, and forming a cell line or a non-human transgenic animal from the selected embryonic stem cell.

The invention further provides a library of embryonic stem cells wherein a multiplicity of cells in the library each contain a gene having inhibited expression, a sequence of the gene having inhibited expression is known, and a multiplicity of different inhibited genes is represented in the library.

The invention further provides a method of creating a library of embryonic stem cells wherein a multiplicity of cells in the library each contain a gene having inhibited expression, a sequence of the gene having inhibited expression is known, and a multiplicity of different non-functional genes is represented in the library, comprising introducing into an embryonic stem cell a vector having a selectable marker which, when the vector is inserted within a gene, the inserted vector can inhibit the expression of the gene, selecting embryonic stem cells expressing the selectable marker, excising the vector from the embryonic stem cells expressing the selectable marker such that host DNA from the gene is linked to the excised vector, sequencing the host DNA linked to or in the excised vector; thereby identifying sequence of the gene whose expression is inhibited, and creating a library of embryonic stem cells containing the gene whose expression is inhibited and a sequence of the inhibited gene is known.

The invention further provides a method of selecting a cell line or a non-human transgenic animal model for the analysis of the function of a gene comprising introducing into an embryonic stem cell a vector having a selectable marker which, when the vector is inserted within the gene, the inserted vector can inhibit the expression of the gene, selecting embryonic stem cells expressing the selectable marker, excising the vector from the embryonic stem cells expressing the selectable marker whereby host DNA from the gene is linked to the excised vector, sequencing host DNA in the excised vector, comparing the sequence of the host DNA to known gene sequences to determine which host DNA is from a gene for which a model for the analysis of the function the gene is desired, and selecting the embryonic stem cell containing the inhibited gene for which a model for the analysis of gene function is desired.

### BRIEF DESCRIPTION OF THE DRAWINGS

FIG. 1 shows the strategy for tagged sequence mutagenesis. The U3NeoSV1 gene trap retrovirus shuttle vector contains coding sequences for a neomycin resistance gene (Neo) located in the long terminal repeats (LTRs) at each end of the provirus. Selection for neomycin resistance generates ES cell clones in which expressed cellular genes have been disrupted as a result of virus integration. This occurs when the promoter of the disrupted gene activates expression of the Neo gene in the 5′ (leftward) LTR. The vector contains a plasmid origin of replication (Ori) and an ampicillin resistance gene (Amp R), allowing portions of the disrupted genes to be cloned by plasmid rescue, as shown. The region immediately adjacent to each provirus is sequenced by using a primer complementary to Neo (NeoC primer: 5′-ATCTTGTTCAATCATGCG-3′ (SEQ ID NO. 1)). This generates a unique sequence tag (PST) for each insertion mutation that is used to identify genes disrupted in individual ES cell clones.

FIG. 2 shows the distribution of PST BlastN scores. PSTs from a library of 400 ES cell clones were compared to the non-redundant GenBank database by using the BLASTN program, and the distribution of scores from all searches is plotted. Approximately 10% of the PSTs matched previously characterized genes (Table 1) or ESTs (Table 2), and scores for these matches are shown in black. The remainder did not match identifiable genes.

FIG. 3 shows progressive identification of genes disrupted by tagged sequence mutagenesis. The ability to identify genes disrupted in a library of 400 ES cell clones has increased dramatically as the nucleic acid databases have expanded in size. Known genes are shown in black while the contribution of anonymous cDNAs and ESTs are shown in white. The total number of genes matching sequences in the catalog of PSTs has increased 3150 percent over the past 8 years.

FIG. 4 shows functional genomics by tagged sequence mutagenesis. Gene Discovery: Cloned cDNAs are compared to NCBI nucleic acid databases using the BLAST algorithm (http://www.ncbi.nlm.nih.gov/). Coding sequences for an unknown gene are likely to be represented in the EST databases as anonymous cDNAs. For the purpose of illustration, applicant queried cDNA sequences for the known gene α-NAC. The search revealed 311 ESTs, which could be overlapped with each other to form a cDNA contig and span the entire α-NAC mRNA transcript. A search of the non-redundant database revealed the identity of the gene as α-NAC, which has two splice forms. Mutant Identification: The complete cDNA contig is compared to the PST database (to be included in the Genome Survey Sequence (gss) NCBI database). This contig matched exon sequences in two PSTs, termed E24U, and E69R, identifying insertion mutations in the corresponding ES cell lines. Gene Function: The E24U and E69R disruption mutations of the α-NAC gene are immediately available for transmission into the mouse germline. Generation of mice homozygous for each mutation can then be used for phenotypic analysis and as a source of cell lines for biochemical studies. Gene Structure: In addition to their usefulness in the functional analysis of α-NAC, the corresponding E24U and E69R rescued plasmids possess several kb of flanking cellular DNA for gene structure analysis. Further genomic sequence can be plasmid rescued using alternative restriction enzymes. In the case of α-NAC, a single BamHI 3′ rescue of either E24U or E69R ES cell lines would yield the remainder of the gene locus. In addition to rapidly cloning the 129 allele of α-NAC, sequence analysis would reveal intron/exon boundaries, transcriptional regulatory elements, and a third PST mutation, M12U, which has been identified by intron sequence. For the α-NAC schematic, coding exons are shown as solid boxes, non-coding exons as open boxes, and the muscle-specific coding exon as a hatched box. The oval depicts a putative promoter and transcriptional initiation site. M12U, E24U, and E69R rescued genomic DNAs are depicted as solid bars at the top of the figure and the known structure of the α-NAC gene is drawn to scale beneath. The dashed lines indicate flanking genomic DNA of unknown lengths; restriction sites are indicated as H, HindIII; S, StuI; R, EcoRI; X XhoI; and B, BamHI.

### DETAILED DESCRIPTION OF THE INVENTION

The present invention may be understood more readily by reference to the following detailed description of the preferred embodiments of the invention and the Example included therein and to the Figures and their previous and following description.

Before the present compounds, compositions, and methods are disclosed and described, it is to be understood that this invention is not limited to specific libraries, specific cell types, specific methods for extracting vectors from host cells, specific conditions, specific selectable markers, or other specific methods, as such may, of course, vary, and the numerous modifications and variations therein will be apparent to those skilled in the art. It is also to be understood that the terminology used herein is for the purpose of describing particular embodiments only and is not intended to be limiting.

As used in the specification and in the claims, “a” or “an” can mean one or more, depending upon the context in which it is used. Thus, for example, reference to “an embryonic stem cell” can mean that at least one embryonic stem cell can be utilized.

In accordance with the purpose(s) of this invention, as embodied and broadly described herein, this invention, in one aspect, provides a method of producing a selected cell line or a non-human transgenic animal model for the analysis of the function of a gene comprising introducing into an embryonic stem cell a vector having a selectable marker which, when the vector is inserted within a gene, the inserted vector can inhibit the expression of the gene, selecting embryonic stem cells expressing the selectable marker, excising the vector from the embryonic stem cells expressing the selectable marker such that host DNA from the gene is linked to the excised vector, sequencing the host DNA in the excised vector, comparing the sequence of the host DNA to known gene sequences to determine which host DNA is from a gene for which a model for the analysis of the function the gene is desired, selecting the embryonic stem cell containing the inhibited gene for which a model for the analysis of gene function is desired, and forming a cell line or a non-human transgenic animal from the selected embryonic stem cell.

By “function of a gene” is meant the biological or physiological role the gene or the gene product has in the host cell and host organism. For example, the gene could have a role such as producing or encoding an RNA molecule that is not translated into a protein or polypeptide, such as a tRNA, a small nuclear RNA (snRNA) or a small cytoplasmic RNA (scRNA). Alternatively, the gene could encode an RNA that is ultimately translated and thereby producing a protein or polypeptide. By inhibiting the expression of the gene, by inhibiting the transcription of the gene, the translation of the RNA transcribed from the gene, or both, one can study or analyze the role or the function of the gene in the cell or host by studying or analyzing the effect of the absence of the normal gene product, whether that normal gene product is an RNA or a protein. The expression of the gene can also be affected by less direct effects as well. For example, the stability of an RNA or a protein can be altered, the ability of the RNA to be transported from the nucleus to the cytoplasm could be affected. The post-transcriptional and/or post-translational processing of an RNA and/or a protein can also be affected that would affect the stability or the activity of the RNA or protein. An effect on the expression of a gene can therefore include these different types of alterations, and the effect of the alteration can be the subject of the analysis of the function of a gene.

As used herein, the term “gene” includes a unit of heredity that occupies a specific locus on a chromosome as well as any sequences associated with the expression of that nucleic acid. For example, a gene includes any introns normally present within the protein coding region as well as non-coding regions preceding and following the coding region. Examples of these non-coding regions include, but are not limited to, transcription termination regions, promoter regions, enhancer regions, modulation regions such as the Glucocorticoid Modulatory Element, receptor binding regions such as a GRE, and the non-transcribed regions between a promoter and the transcription initiation point, and the non-transcribed region between the site or sites of poly(A) addition and the point or region where transcription terminates. Therefore all regions of a host genome that have at least some cis influence on the expression of a region of the genome which is translated into an RNA, or which is transcribed into an RNA which is then translated into a protein or polypeptide, are part of a “gene.”

The inhibition of the gene can be achieved in any number of ways apparent to one skilled in the art, including the insertion of the vector into the gene. This insertion can, for example, result in a frame-shift mutation in the coding region of the gene or an exon of the gene which may result in a truncated protein whose function is inhibited, whether that function is catalytic, structural, or otherwise. Additionally, inhibition of the gene can occur, for example, by insertion of a vector into a non-coding region of a gene, such as adjacent to or within a promoter, adjacent to or within an enhancer, adjacent to or within an RNA processing signal, adjacent to or within a regulatory element binding or response site, and so on, whereby the insertion disrupts or inhibits the transcription of the gene, and/or the translation of the RNA transcribed from the gene. One skilled in the art will appreciate that the inhibition of the function of the gene can occur by many mechanisms and the inhibition is, of course, not limited to any specific example of the specific inhibition of the function of a gene which may result from the insertion of a vector into the gene.

The inhibition of the function of a gene does not have to be a total or complete inhibition of the gene, but the inhibition is preferably to a degree that the normal product of the gene is either not present in an amount to sustain the typical or normal role of the gene product in a cell or host, or is not active to a degree to sustain the typical or normal activity in a cell or host, which therefore allows one to analyze, study, examine, or otherwise determine the effect of the inhibition of the gene upon the cell or the host.

The vector used to inhibit the expression of a gene can comprise any vector capable of inserting into the genome of an embryonic stem cell, preferably a murine embryonic stem cell or a human embryonic stem cell. The vector can therefore comprise a transposon, or a fragment or derivative thereof, which is capable of being inserted or inserting itself into the genome of a cell. Alternatively, the vector can comprise a viral vector, or a fragment or derivative thereof. The vector can comprise an episomal nucleic acid that can be modified to allow insertion of the nucleic acid into the genome of the host. Preferably, the vector is a viral vector whose genome can be inserted into the genome of a cell, and the viral vector is preferably a retrovirus vector. The example provided herein disclosed the use of a retroviral vector which can be used to inhibit the function of a gene. The vector preferably contains sequences which allow the vector to become inserted into the genome of a cell and then not spontaneously excise itself from the genome of the cell. Therefore the integration is preferred to be a stable integration or insertion. It is also envisioned herein, however, that the vector may be excised from the genome of the cell. For example, by culturing the cell under conditions such that the vector is excised from the genome, such as a vector containing a temperature sensitive mutation, or where the vector is excised from the genome of the cell by adding a compound or composition to the cell containing the inserted vector, the integrated vector can be excised from the genome of the host or cell. For example, a nucleic acid sequence which acts in trans to enable the inserted vector to become excised from the genome can be introduced into the cell, or a protein necessary for the excision of the vector from the genome may be supplied to the cells, such that the added sequence or protein complements a sequence or protein of the vector and/or of the cell whereby the vector becomes excised from the genome of the cell. One skilled in the art will appreciate that such controlled excision from the vector from the genome of a cell will provide an additional experimental control for the cell stably containing the vector in its genome.

The vector preferably contains a selectable marker which can be used to screen for those cells which contain the vector in their genome and which express the selectable marker. In this manner, one can readily separate those cells containing the vector and expressing the selectable marker from those cells either containing the vector but not expressing the selectable marker, and from those cells not containing the vector. The specific selectable marker used in the vector can of course be any selectable marker which can be used to select against eukaryotic cells not containing and expressing the selectable marker. The selection can be based on the death of cells not containing and expressing the selectable marker, such as where the selectable marker is a gene encoding a drug resistance protein. An example of such a drug resistance gene for eukaryotic cells is a neomycin resistance gene. Cells expressing a neomycin resistance gene are able to survive in the presence of the antibiotic G418, or Geneticin®, whereas those eukaryotic cells not containing or not expressing a neomycin resistance gene are selected against in the presence of G418. One skilled in the art will appreciate that there are other examples of selectable markers, such as the hph gene which can be selected for with the antibiotic Hygromycin B, or the E. coli Ecogpt gene which can be selected for with the antibiotic Mycophenolic acid. The specific selectable marker used is therefore variable.

The selectable marker can also be a marker that can be used to isolate those cells containing and expressing the selectable marker gene from those not containing and/or not expressing the selectable marker gene by a means other than the ability to grow in the presence of an antibiotic. For example, the selectable marker can encode a protein which, when expressed, allows those cells expressing the selectable marker encoding the marker to be identified. For example, the selectable marker can encode a luminescent protein, such as a luciferase protein or a green fluorescent protein, and the cells expressing the selectable marker encoding the luminescent protein can be identified from those cells not containing or not expressing the selectable marker encoding a luminescent protein. Alternatively, the selectable marker can be a sequence encoding a protein such as chloramphenicol acetyl transferase (CAT). By methods well known in the art, those cells producing CAT can readily be identified and distinguished from those cells not producing CAT.

The vector can be introduced into the embryonic stem cell using any of a number of methods or procedures. For example, and as described in the Example contained herein, the vector can be a defective retrovirus, such as a defective Moloney leukemia virus, which can be packaged into a virus particle capable of infecting an embryonic stem cell. This virus can then infect an embryonic stem cell and thereby deliver the genome of the virus to the cell. Alternatively, the vector can be introduced directly into the embryonic stem cell by techniques such as calcium phosphate transfection, liposome delivery, DEAE-dextran mediated transfection, lipofectin-mediated transfection, injection, cell or protoplast fusion, electroporation, or by using non-viral based vectors that are able to introduce a nucleic acid into the genome of an embryonic stem cell.

Once the vector has been introduced into the embryonic stem cell, that vector, or a fragment thereof, can then be excised from the genome of the embryonic stem cell. As described in the Example contained herein, the genome of the embryonic stem cell containing the vector can be digested with a restriction enzyme such that a nucleic acid fragment produced by the digestion contains at least part of the vector which is capable of being identified, such as a fragment containing a sequence not present in the genome of the embryonic stem cell (i.e a “sequence tag” or a “tagged sequence”), and part of the genome from the embryonic stem cell. Alternative, the vector, or a fragment thereof, can be excised from the genome of the embryonic stem cell by physically shearing the genome of the embryonic stem cell containing the vector. Alternatively, the vector, or a fragment thereof, can be excised from the genome of the embryonic stem cell containing the vector by using a compound or composition, such as a helper virus, whereby the vector, or a fragment thereof, is excised from the genome of the embryonic stem cell containing the vector and part of the genome from the embryonic stem cell. The precise method of excising the vector, or a fragment thereof, including at least part of the genome of the embryonic stem cell, from the genome of the embryonic stem cell containing the vector can vary, but the resulting nucleic acid fragment comprising the vector, or a fragment thereof, should preferably contain part of the genome from the embryonic stem cell. This part of the genome from the embryonic stem cell would be linked to the nucleic acid comprising the vector, or a fragment thereof, such that the position of the part of the genome with respect to the nucleic acid comprising the vector, or a fragment thereof, remains stable, unless manipulated to be otherwise. Therefore the part of the genome of the embryonic stem cell can be covalently linked to the vector, or a fragment thereof, or otherwise, just so that the respective parts remain positionally stable. For example, the nucleic acid comprising the vector, or a fragment thereof, can be linked to the part of the genome of the embryonic stem cell by complementary overhangs on the termini of the nucleic acids. Any gap in the overhangs, or any nick in the overhangs, can be repaired, if necessary, by treating the nucleic acids with appropriate enzymes together with the other necessary components such as salts, buffer, nucleotides, cofactors, and so on, or the gap and/or nick can be repaired by introducing the linked nucleic acids into a cell which can thereby repair the gap and/or nick.

One skilled in the art will appreciate that typically not all the embryonic stem cells will have the vector excised, but some of the cells will be maintained with the vector remaining in the genome of the cell so that one can then have the embryonic stem cell containing the vector which inhibits a gene available for later manipulations or analysis. In such a manner, a library of embryonic stem cells containing a vector, preferably where the vector contains a selectable marker whose expression is directed by a promoter of a gene of the embryonic stem cells, can be obtained and/or maintained.

One skilled in the art will also appreciate that the embryonic stem cells containing a vector can be cultured under conditions such that cell lines of cells containing a vector in the same position of the genome of the cell can be isolated and maintained. For example, the cells containing the vector and expressing the selectable marker can be diluted in wells of a culture dish such that each well contains no more than a single cell which proliferates. The cell can then be allowed to proliferate and the cell lines resulting from such manipulative steps should be at least relatively pure cell lines. This, therefore, provides another way in which a library of embryonic stem cells containing a vector can be produced and/or maintained. Once a sequence from part of a gene of the embryonic stem cell is identified and selected for analysis of the function of the gene, one can rapidly obtain a cell from such a population or library for further manipulation that contains a vector inserted within or adjacent to, and thereby inhibiting, the gene of interest.

Alternatively, the embryonic stem cells containing the vector and expressing the selectable marker can be maintained as a mixed population until a sequence of a gene of the embryonic stem cell is determined and chosen for analysis of the function of the gene, and the cell containing a vector at the same position of the same gene can be isolated from the mixed population.

The vector can also contain other sequences or regions that by the presence of the sequence or region itself, or through a product encoded by the sequence or region, functions to assist or enhance the isolation of excised nucleic acid fragment comprising the vector, or a fragment thereof, and part of the genome from the embryonic stem cell. For example, part of the vector which is excised from the genome of the embryonic stem cell can be a sequence that is capable of being selectively or specifically bound by a protein or antibody. One example of such an enhancement sequence is the lac operator (lac O), which can be bound by the lac repressor. One skilled in the art will appreciate that a lac repressor can be linked to another protein such as β-galactosidase, and when the lac repressor/β-galactosidase fusion protein binds to the lac O region of the vector, that bound complex can be isolated from the remaining components in a mixture by binding the lac repressor/β-galactosidase fusion protein-lac O complex to anti-β-galactosidase antibodies which may be immobilized on a substrate, such as magnetic beads, to capture or selectively bind the complex while the remaining components of the mixture are removed. One example of a reagent for the isolation of β-galactosidase fusion proteins is the ProtoSorb lac Z immunoaffinity absorbent. (Promega Corp.).

Where a vector contains a selectable marker, it is preferable that the vector does not contain a promoter that can direct expression of the selectable marker in an embryonic stem cell. The vector can therefore contain a promoter per se, but that promoter would not direct or promote transcription of the sequence encoding the selectable marker when in an embryonic stem cell. For example, a promoter could be positioned 3′ to the sequence encoding the selectable marker, or the promoter could be positioned 5′ to the sequence encoding the selectable marker but the promoter could be functionally inactive in the embryonic stem cell. Regardless, the expression of the selectable marker in the vector, when introduced into an embryonic stem cell, is directed, driven, or promoted by a promoter of the embryonic stem cell. Therefore, where an embryonic stem cell contains such a vector, the expression of the selectable marker would require the vector insert into the genome of the embryonic stem cell in a position such that a promoter within the genome of the embryonic stem cell would be required to direct expression of the selectable marker. Using such a vector, one can therefore effectively enhance the probability of obtaining an embryonic stem cell which expressed the selectable marker wherein the selectable marker of the vector is operatively linked to a promoter of the embryonic stem cell. One can therefore increase the probability that when the vector is excised from the embryonic stem cell, the part of the genome of the embryonic stem cell that is linked to the excised vector, or fragment thereof, will contain at least part of a promoter of a gene of the embryonic stem cell, and/or a region adjacent to a promoter of the embryonic stem cell.

The vector can also contain a non-mammalian origin of replication which can be used to replicate the excised nucleic acid fragment comprising the vector, or a fragment thereof, in another cell such as a bacterial or yeast cell. Therefore excising the vector from the genome of the embryonic stem cell containing the vector can include a technique such as “plasmid rescue.” By having this non-mammalian origin of replication, one can therefore replicate the nucleic acid comprising the vector, or a fragment thereof, in a non-mammalian host to maintain a stock of the fragment, which may then be used for other purposes, such as nucleic acid sequencing, gene mapping, generating hybrid cells, and so on. It will be apparent to one skilled in the art that the nucleic acid fragment introduced into a non-mammalian cell replication host can be selectively maintained and identified by using a selectable marker, or an antibiotic resistance gene, present on the nucleic acid fragment that can be functionally used in the non-mammalian replication host cell. For example, ampicillin resistance can be used to select and/or maintain those prokaryotic cells expressing a nucleic acid encoding a β-lactamase protein. The invention, therefore, also provides replication hosts containing a nucleic acid comprising a vector, or a fragment thereof, linked to at least part of the genome from an embryonic stem cell.

Once the nucleic acid fragment comprising the vector, or a fragment thereof, and at least part of the genome of the embryonic stem cell is excised from the embryonic stem cell containing the vector, at least part of the genome of the embryonic stem cell which is linked to the nucleic acid fragment comprising the vector, or a fragment thereof, can be sequenced. The nucleic acid sequence can be derived by many techniques well known in the art, such as direct PCR sequencing, subcloning the fragment followed by sequencing, such as in M13 sequencing procedures, or even by transcribing DNA into RNA and then performing RNA sequencing. Regardless of the specific method used to determine the sequence of at least part of the genome of the embryonic stem cell which is linked to the vector, of a fragment thereof, that information can ultimately be used to determine the sequence of part of a gene of the embryonic stem cell since the part of the genome of the embryonic stem cell which is linked to the vector, or a fragment thereof, would be derived from a promoter of a gene of the embryonic stem cell, or an adjacent sequence.

Once the sequence information is obtained, the different individual cells or cell lines derived or produced from the embryonic stem cells containing a vector therefore provide a library of embryonic stem cells wherein a multiplicity of cells in the library each contain a gene having inhibited expression, a sequence of the gene having inhibited expression is known, and a multiplicity of different inhibited or non-functional genes is represented in the library. In a preferred embodiment, the majority, and more preferably, substantially all of the embryonic stem cells contain a single gene having inhibited expression. In addition, in a preferred embodiment a majority of the embryonic stem cells of the library contain different genes having inhibited expression. More preferably, the library contains a majority of the expressed genes with inhibited expression.

The library can be produced or created using the methods described herein. The vector in the embryonic stem cells containing a vector preferably contains a selectable marker and an origin of replication which will allow an excised vector to replicate in a replication host. The origin of replication is preferably non-mammalian, and can include yeast and prokaryotic origins of replication.

This sequence information can then be compared to known sequences in databases such as GenBank, to determine whether the nucleic acid corresponds to a known gene whose function is unknown, or to a previously unknown gene, whose function is therefore also unknown. Even those genes whose function is known, but for example, the mechanism of action or the pathway location of a protein encoded by the gene has not been conclusively determined, may be chosen for further analysis or examination. An example of a comparison of the sequence of part of a gene from an embryonic stem cell, obtained from a vector insertion method as described herein, to known sequences is disclosed in the Example included herein.

The present invention therefore also provides a method of selecting a cell line or a non-human transgenic animal model for the analysis of the function a gene comprising introducing into an embryonic stem cell a vector having a selectable marker which, when the vector is inserted within the gene, the inserted vector can inhibit the expression of the gene, selecting embryonic stem cells expressing the selectable marker, excising the vector from the embryonic stem cells expressing the selectable marker whereby host DNA from the gene is linked to the excised vector, sequencing host DNA in the excised vector, comparing the sequence of the host DNA to known gene sequences to determine which host DNA is from a gene for which a model for the analysis of the function the gene is desired, and selecting the embryonic stem cell containing the inhibited gene for which a model for the analysis of gene function is desired.

Once the sequence of part of a gene from an embryonic stem cell has been determined and selected for analysis of the function of the gene, the cells containing the vector located within, and inhibiting the gene, can be used to generate or form a cell line or a non-human transgenic animal.

Using protocols known in the art, embryonic stem cells can be maintained on feeder cell layers in a medium containing appropriate growth hormones to inhibit their differentiation, as described in Hogan, BLM “Pluripotential Embryonic Stem Cells and Methods of Making Same”, U.S. Pat. No. 5,453,357 Issued Sep. 26, 1995. Feeder cells are preferably derived from murine embryos, but feeder cells from any animal species and any tissue thereof are also contemplated. Media that maintain ES cells in an undifferentiated state in the absence of feeder cell layers are also contemplated.

Cultured embryonic stem cell lines can be allowed to differentiate in vitro into any number of cell and tissue types, including but not limited to: trophoblast, endoderm, embryonic ectoderm, myocardium, epithelium, skeletal muscle cells, neural cells, and fibroblasts. One method to allow in vitro differentiation is to culture the embryonic stem cells in the absence of feeder cell layers and growth hormones that inhibit differentiation (see, for example, Graves and Moreadith, 1993, Mol. Reprod. Dev. 36:424-433; Notarianni et al., 1990, J. Reprod. Fert (Suppl.) 41: 51-56; and Notarianni, et al. 1991, J. Reprod. Fert (Suppl.) 43: 255-260).

Transgenic animals can be derived from embryonic stem cells or embryonic stem cells in which a vector is inserted into the genome of the cell and inhibited the expression of a gene by any of a number of techniques known in the art, including but not limited to chimeric embryo formation (see, for example, Labosky et al., 1994, Development 120:3197-3204; Giles et al., 1993, Mol. Reprod. Dev. 36:130-138) or ES cell nuclear transfer to an enucleated oocyte (see, for example, Sims and First, 1993, Proc. Natl. Acad. Sci 90:6143-6147; Campbell et al., 1996, Nature 380:64-66; Stice et al., 1996, Biol. Reprod. 54:100-110). Transgenic animals so derived can be studied directly to discern function of the inhibited gene, or in the case of chimeric animals, these animals can be bred with other animals of the species to derive non-chimeric, fully transgenic animals. Such animals, as well as transgenic animals created through nuclear transfer, can then be studied directly to discern the function of the inhibited gene, and they can be further bred with other animals of the species to determine phenotype dominance and to identify complementing mutations. Finally, embryos derived from transgenic animals can be used to generate new embryonic stem cell lines, following procedures well known in the art (see, for example, Hogan, B L M, U.S. Pat. No. 5,453,357; Evans and Kaufman, Nature 292:154-156; Robertson, E J (1987), “Teratocarcinomas and embryonic stem cells—A practical approach”, London: IRL Press Oxford, pp. 71-112).

### EXAMPLE

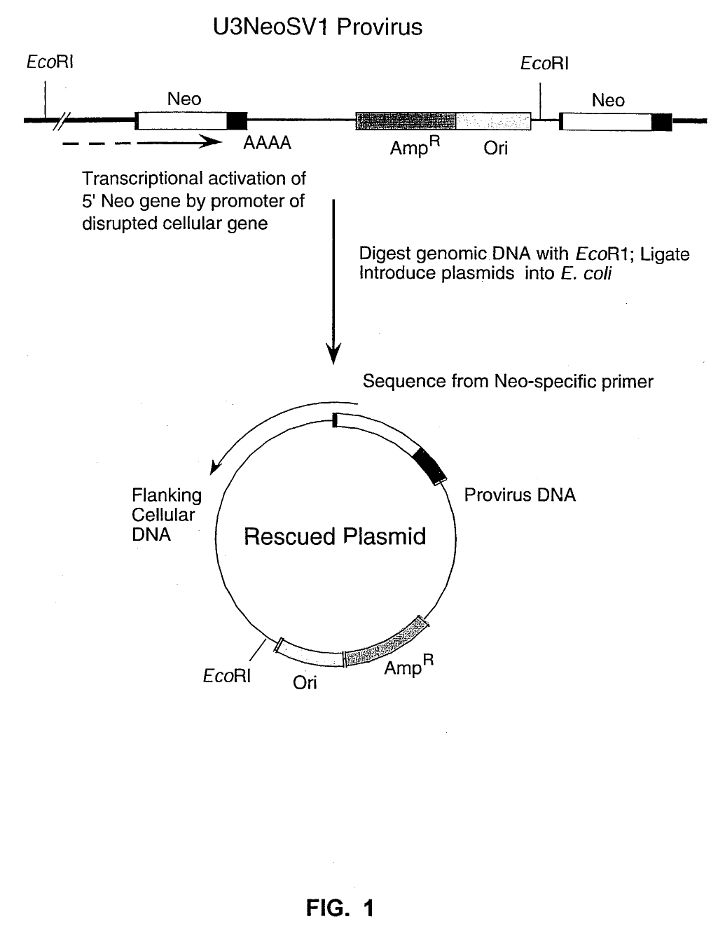

The strategy of tagged sequence mutagenesis is shown in FIG. 1. A gene trap retrovirus shuttle vector, U3NeoSV1, was developed to generate a library of embryonic stem (ES) cell clones, each containing a single gene disrupted by virus integration. The U3NeoSV1 virus carries a promoterless neomycin resistance gene in the U3 region of the long terminal repeat (LTR). While retroviruses integrate widely throughout the genome (18, 19), neomycin resistance selects for those cells in which the virus has inserted into expressed cellular genes (FIG. 1). A pBR322 plasmid origin of replication and an ampicillin resistance gene in the vector allow DNA sequences flanking the provirus to be cloned directly in E. coli.

ES cell colonies expressing the neomycin resistance gene (Neo^{R}) were cloned and expanded in mass culture. Early passage cells were cyropreserved and used to prepare genomic DNA. To clone flanking cellular sequences, 5 μg of genomic DNA was digested with EcoRI, ligated under conditions to promote circularization, and electroporated into E. coli. The identity of each rescued plasmid was confirmed by Southern blot hybridization, comparing the size of the cloned EcoR1 fragments with the corresponding genomic DNAs. The mean size (+SD) of the rescued plasmids was 7.8±4.4 Kb and the largest was 23 Kb. This is similar to the distribution of fragment sizes of EcoRI digested genomic DNA.

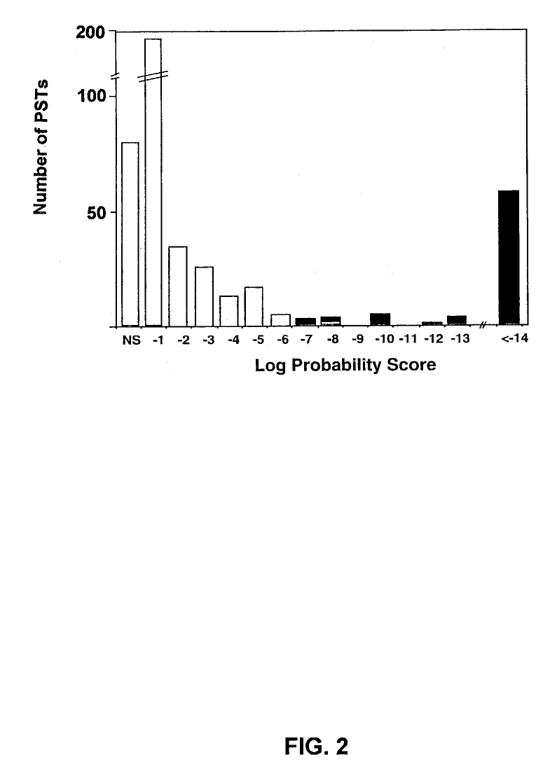

Regions of genomic DNA adjacent to each provirus were sequenced, extending (±SD) an average of 297+71 nucleotides from a single Neo-specific primer (FIG. 1). This provided a unique sequence tag for each insertion mutation, which we designated, “Promoter-proximal Sequence Tags” or PSTs. The PSTs were compared to the non-redundant GenBank database by using the BLASTN program (20). This program searches for stretches of nearly identical sequence, and matches are scored according to the probability of their occurrence by chance alone. The scores from all searches, excluding matches with repetitive sequences, are summarized in FIG. 2. In 42 cases (approximately 10% of PSTs) the search revealed specific genes disrupted as a result of provirus integration (Table 1), and 21 additional targets matched anonymous cDNAs present in the dbEST database (Table 2). It is significant that the majority of matching ESTs were derived from murine cDNAs since human ESTs far outnumber mouse ESTs in dbEST. Human cDNAs, particularly 5′ exons, probably lack sufficient sequence identity to match PSTs when compared by using BlastN. The addition of increasing numbers of murine ESTs to the databases should greatly enhance the identifications of gene sequences disrupted by tagged sequence mutagenesis.

All known targeted genes were unambiguously identified according to several criteria. First, the probability scores were highly significant, generally ranging from 10⁻⁹ to 10⁻⁹³, due to stretches of nearly identical sequence. Most matches involved cDNAs and ended abruptly at 5′ or 3′ consensus splice sites, depending on whether the virus integrated into an exon or an intron. Thus, the range of scores primarily reflects the amount of exon in each PST rather than the overall sequence similarity. Second, matches involving these genes generated scores significantly lower than any other match with the same PST (Table I). This eliminates matches that might result from families of related sequences. Third, each provirus was in the same transcriptional orientation as the target gene and was typically located toward the 5′ end of the gene.

These results provide molecular information relevant to the broader use of gene entrapment in genetic studies. The disrupted genes are all transcribed by RNA polymerase II and except GLUT1 and a gene linked to Ly-6E, contain proviruses inserted within 350 nt. of an exon. 16 inserts listed in Table 1 were in exons, and 10 were positioned upstream of the initiation codon of the disrupted gene. The average cell-virus fusion transcript is predicted to contain approximately 500 nt. of cellular RNA, in agreement with Northern hybridization studies (10, 13, 15).

Nearly 85% of PSTs examined represent previously uncharacterized gene sequences and failed to generate any significant matches. Most had probability scores of 0.1 or larger (FIG. 2), although a few returned scores as low as 10⁻⁸. These latter matches did not involve cognate genes according to the criteria listed above, but may reflect functionally related elements. Nevertheless, the ability to identify genes among the catalog of sequence tags appears to be limited primarily by the number of characterized genes in the nucleic acid databases. Thus, the proportion of PSTs matching known genes is similar to the representation of known genes among non-redundant ESTs (21-24) and among genomic DNA sequences recovered after exon trapping 25. Even so, some target genes may be missed because the flanking DNA lacks sufficient exon sequences to generate a statistically significant score. This could occur if the provirus inserted near a promoter or splice acceptor site or further within an intron.

The efficiency of tagged sequence mutagenesis will allow many of the estimated 10,000-20,000 genes expressed in ES cells to be disrupted and characterized within the next few years. Once completed, the biological functions of a large number of genes can be assessed without having to characterize the genomic structure of the gene or to target the gene by homologous recombination. This is important because most mammalian genes are identified by methods that reveal little about their biological functions. For example, among genes disrupted in the present study: (i) FUS and EWS are translocated in human solid tumors (26-29); (ii) plk and NonO are homologues of genes responsible for mutant phenotypes in Drosophila (30-35). (iii) FBP binds DNA sequences upstream of the c-myc promoter (36); and (iv) Gas5 is differentially expressed in growth arrested cells (37).

Finally, libraries of mutant clones will also permit new types of genetic analyses. In particular, it will be possible to screen for specific phenotypes after introducing a number of mutations into the germline. Subsequent studies can then focus on those genes that are important to a specific biological problem. For example, the identity of RNA binding proteins that regulate the expression of specific cellular genes can be determined. Such proteins are expected to influence tissue-specific phenotypes, whereas, mutations affecting basic metabolic processes such as splicing or RNA transport should result in early embryonic death.

Functional Analysis of Genes Identified by PSTs

To date 16 mutations induced by U3gene trap vectors have been introduced into the germline, of which six resulted in obvious phenotypes when bred to a homozygous state. Recessive lethal phenotypes have resulted even when the virus integrated into an alternatively spliced, 5′ non-coding exon of the Ran GTPase activating Protein (Fug1) (38) and into introns of genes encoding hnRNP U, hnRNP C and a protein methyl transferase. Insertions in Fug1, hnRNP C and the Eck receptor tyrosine kinase caused null mutation (38, 39); however, at least one insert, in an intron of the hnRNP A2/B1 gene, failed to ablate gene expression. Thus, gene trap mutagenesis usually disrupts gene function; and in cases where the consequences of provirus insertion are uncertain, the mutations can be evaluated at the nucleotide level prior to germline transmission. Further, as libraries of insertion mutations approach saturation, it is expected that PSTs will identify multiple insertions into the same target gene.

Number and Types of Gene Targets

The number of genes in the genome that can be disrupted by gene trap selection was previously estimated, firstly, from the fraction of proviruses that express U3 genes and, secondly, by the frequencies with which single-copy genes are disrupted following gene trap selection (18). In each case, the estimated number of gene targets (2-10×10⁴) was comparable to the total number of expressed genes as determined by RNA renaturation kinetics (40). The number of gene targets identified in Tables 1 & 2, quadruple the number of genes characterized by all previous gene entrapment studies (10-12, 14, 38, 39, 41-44). The number and complexity of these genes suggest -that a large number of genes can be targeted. Finally, the frequency of LINE-1 and VL30 inserts is similar to the relative abundance of these multicopy transcription units in the mouse genome (45).

Two genes, L29 and α-NAC, were disrupted multiple times (three times each). This suggests that mutagenesis by U3NeoSV1 is not entirely random. For comparison, there is a 50-50 chance that 2 of 400 inserts will disrupt the same gene assuming there are 10,000 potential target genes and that gene entrapment is entirely random. It is possible that either integration or selection for U3 gene expression will be skewed in favor of certain genes. For example, factors affecting translation of the resulting fusion transcript should affect the size of the region with in a gene that allows neo expression. These would include sequences affecting translation of the downstream Neo reading frame. Strong promoters could compensate for inefficient translation allowing expression of proviruses inserted further within the gene. Finally, factors affecting the definition of U3 Neo sequences as a 3′ terminal exon affect the expression of U3 Neo genes inserted into introns. However, retrovirus integration appears to occur throughout much of the genome 46, 47, and the process appears remarkably random (19).

Nevertheless, no mutagen is entirely random, including simple alkylating agents. The possibility that some genes may be targeted more easily than others is not expected to have a serious impact on tagged sequence mutagenesis given the ease of analyzing large numbers of mutations. However, it may be possible to understand factors responsible for preferential targeting, which in turn may shed light on genome structure, organization and function.

PSTs as Expressed Sequence Tags

PSTs represent the first expressed sequence tags derived from genomic DNA, and as such, they define functional and structural features of genes missing from cDNA sequences. Consequently PSTs will complement the use of ESTs in genome research. First, transcriptional promoters are frequently present in the larger rescued plasmids from which PSTs are derived. These include 14 presumptive promoter regions (i.e. extensive sequences upstream of the 5′ end of published cDNAs) for genes listed in Table 1. Second, intron/exon boundaries can be determined by aligning PST and EST sequences. Among PSTs matching known genes (Table 1) 14 and 23 included 3′ and 5′ splice sites, respectively. Third, gene entrapment is less biased for highly expressed genes than is cDNA cloning, providing more uniform gene representation. For example, 10% of ESTs from brain are related to cytoskeletal proteins; whereas, none of the ES cell PSTs match cytoskeletal genes. Only 5 PSTs (Histone H1, L19 ribosome subunit protein, EWS, fau/S30, and the polyA binding protein) were represented among 700 known genes in studies of brain ESTs, and none constituted more than 0.01% of randomly sequenced cDNAs (21-23). Fourth, PSTs enrich for promoter-proximal exon sequences, often under-represented in, cDNA libraries. Fifth, probes derived from PST clones distinguish between transcribed genes and non-expressed pseudogenes. For example, expressed Line-1 elements were identified from among 10⁵ non-expressed segments in the mouse genome (45). Finally, the emerging catalog of PSTs describes the transcriptional repertoire of ES cells—genes which collectively define the unique biological properties of the pluripotent stem cell. For example, while the genomes of early embryos and ES cells are significantly hypomethylated (48, 49), this does not appear to result in widespread derepression of cellular gene expression, as monitored by gene entrapment.

Functional Genomics in Mice

Mice are presently the only mammalian organism suited for large-scale studies of gene function. While other model organisms have unique features that can be exploited for particular purposes, mice are more likely to provide accurate models of human disease. Another unique aspect of using mice as a genetic system is the potential for generating cell lines deficient for specific gene functions with which to analyze biochemical functions of the encoded proteins. For example, null cells have been used to identify the role of the p53 tumor suppressor in cellular responses to anti-cancer therapy and to identify critical target genes regulated by p53 (50-52). The importance of genetically defined cell lines cannot be over-stated, and in this regard the mouse, is superior to other model organisms (e.g. Drosophila, C. elegans, or zebra fish) from which cell lines are not easily obtained. Null cells can be isolated from mice even when the mutation results in early embryonic death. In many cases, null cells can be derived from ES cells without germline transmission (53).

Summary and Future Prospects

In conclusion, this application describes a new paradigm for analyzing mammalian gene function on a large scale. The capacity to induce, characterize and maintain mutations in ES cells circumvents many limitations associated with conventional mammalian genetics. Libraries of sequenced mutations help bridge the increasing gap between gene sequences and their unknown functions, thus facilitating a functional analysis of the mouse genome.

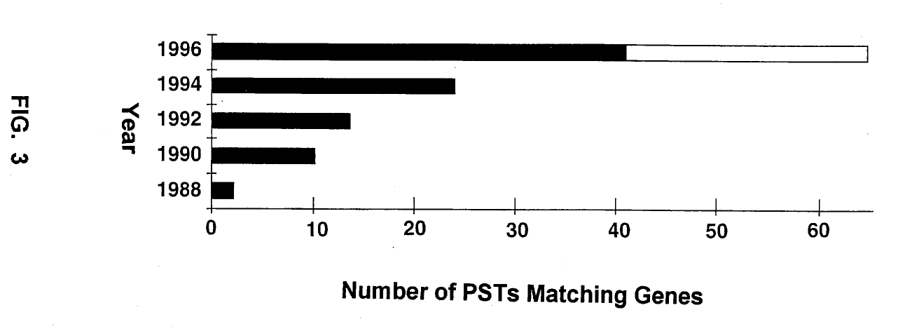

As new genes and gene sequences are characterized, the percent of PSTs expected to identify mutations in known genes should increase significantly in the next few years. As shown in FIG. 3 almost two-thirds of the genes that matched in the screen of 400 PSTs were characterized in the past four years. The number matching anonymous cDNAs should increase at an even faster rate as greater numbers of murine ESTs are added to the databases.

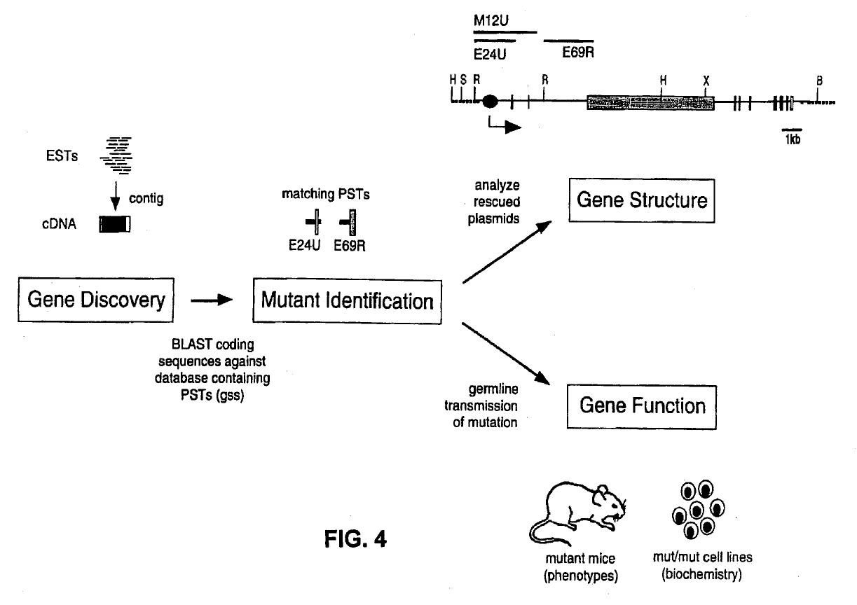

The protein coding sequences for most mammalian genes will discovered as ESTs are assembled into longer contiguous sequences. Mutations can then be selected for germline transmission based on the predicted sequences of the encoded proteins. For example, three inserts in our mutant library occurred in different regions of the α-NAC gene, as shown in FIG. 4. The fact that the genomic sequence of α-NAC is already known (54) helps illustrate how PSTs can be used to analyze gene structure and function. Differentially spliced α-NAC transcripts encode a muscle specific transcription factor and a widely expressed protein associated with signal recognition particle (SRP). The reading frame of the latter protein is completely contained among 311 overlapping ESTs; thus, the PST from E24U cells identifies a mutation within the corresponding protein coding sequence. Another mutation (E69R) disrupts sequences specific to the muscle specific transcript, but the effected protein could not be identified, since only 2 ESTs in the database were derived from this region. While short sequence tags are often sufficient for gene identification, additional information about gene structure can be obtained by sequencing the larger segements of genomic DNA that are recovered by plasmid rescue. The genomic sequences rescued from clones E24U, M12U and E69R span most of the 5′ end of the gene, including portions of three exons and possibly, promoter elements required for tissue specific gene expression.

Tagged sequence mutagenesis complements but does not replace the use of homologous recombination in the analysis of gene function. The effort and expense of direced gene targeting is not suited for screening sets of genes for specific biological activitities. Tagged sequence mutagenesis reduces the effort and expense required to assess loss of function mutations. The resulting phenotypes may then reveal the need to construct other, more subtile mutations or conditional knockouts. Finally, the ability to clone specific regions of genomic DNA, quickly and directly by plasmid rescue could accelerate the construction of specialized vectors for gene targeting by homologous recombination.

In the future, new entrapment vectors and automation, particularly with DNA sequencing, will have an important impact on tagged sequence mutagenesis. Strategies to disrupt non-expressed genes are being developed, and vectors that incorporate site-specific recombination sequences will assist efforts to modify large segments of mammalian chromosomes (55).

Methods and Materials

ES Cells and the U3Neo Shuttle Vector

pRaU3Neo, DNA template for the gene-trap retrovirus shuttle vector U3NeoSV1, was constructed by replacing the BamHI-EcoRI envelope fragment of pGgU3neoen(−)(10) with a shuttle rescue cassette containing the β-lactamase (ampicillin resistance) gene and the low copy number plasmid origin of replication derived from pBR322. Cell lines expressing a packaging-defective ecotropic helper virus (ψ2) were transfected with pRaU3Neo and selected in 400 mg/ml G418. Producer cell lines were titered on NIH-3T3 cells (typically 4×10⁵ cfu per ml per 10⁶ producer cells) as previously described (56).

Mouse embryonic stem cell line ES-D3 cells (129; XY; agouti/agouti) originally derived by Rolf Kemler were the gift of Janet Rossant and Rudolf Jaenisch. ES cells were cultured on irradiated mouse embryo fibroblast layers (MEFs) in high glucose DMEM supplemented with 15% preselected fetal bovine serum (Invitrogen; heat inactivated at 55° C. for 30 min), 100 mM nonessential amino acids (Gibco), 0.1 mM 2-mercaptoethanol, and 1000 units of leukemia inhibitory factor (ESGRO, Gibco) per ml. ES cells are infected with U3NeoSV1 at an MOI of 0.1 by adding 2 ml of diluted and filtered viral supernatant from producer line ψ85 to 10⁵ ES cells (plated 12 h previously on a 15 cm dish) in the presence of 8 μg/ml Polybrene (Sigma). The cells are incubated for 1 hour at 37° C. with occasional rocking, at which time, 18 ml of fresh ES cell medium is added. Allowing 36 h for gene trap selection of expressed cellular genes disrupted by proviral integration, neomycin resistant clones are selected in ES medium containing 300 mg/ml G418 for 7 further days. Individual undifferentiated colonies are then cloned into microtitre dishes and sequentially expanded into two 35 mm dishes, from which one is used for DNA isolation while the remaining cells are cryopreserved in liquid nitrogen.

Plasmid Rescue

Dense monolayers of cloned NeO^{R}ES cells are lysed in tail buffer [100 mM Tris-HCl, pH 8.5; 5 mM EDTA; 0.2% SDS; 200 mM NaCl; 10 mg/ml RNaseA, 200 μg/ml Proteinase K] and cellular DNA extracted as described (57).

10-20 μg of DNA from ES cell clones with a single intact provirus is digested with 50U EcoRI (NEB; high concentration) for 2-3 h in a volume of 250 μl. The digests are heat-inactivated, are allowed to cool to room temperature and purified through a Wizard DNA Clean column as specified by the manufacturer (Promega). The eluate (75 μl) is ligated at a concentration of 5 μg/ml. Samples are heated to 68° C. for 10 min, rapidly cooled on ice, ligase reagents are added at 0° C., and the reactions are incubated overnight at 16° C. Each ligation reaction (0.5 ml) contains: 2.5 μg of EcoRI digested DNA, 50 μl 10× ligation buffer (50 mM Tris 7.6, 10 mM MgCl2, 1 mM DTT), 1.0 mM ATP, and 4.0 Wiess U of T4 DNA ligase (NEB). Following ligation, samples are heat inactivated for 20 min at 68° C., purified over the Wizard columns, precipitated, and resuspended in 5 μl of water.

1.0 μg of ligated DNA (2 μl) is carefully transferred to the inside wall of a prechilled 0.1 cm electroporation cuvette. 25 μl of electro-competent DH10B E. coli cells (GIBCO) is added to the droplet of DNA and electroporation is performed at 200 ohm, 25 μF, and 1.8 KV. Time constants of 4.3 to 4.8 typically give 2×10⁹ to 2×10¹⁰ colonies/μg with supercoiled plasmid controls. 800 μl of SOC is added to electroporated cells within 2 seconds. The bacteria are transferred to a 6 ml tube and incubated for 1 hour at 37° C. with shaking. 400 μl is plated onto a 150 mm LB-Amp (50 μg/ml) dish, and colonies are counted after 16 h at 37° C. The average efficiency of plasmid rescue is 100 colonies/μg of genomic DNA.

DNA Sequencing

5 μg of minipreped plasmid DNA is used in each sequencing reaction as described (58), with the following modifications: (i) a 1:8 dilution of the G-mix was used, (ii) termination reaction uses 1.0 μl of termination and 1.5 μl of extension mix (iii) labeling is for 4 min at room temperature, and (iv) termination is for 5 min at 37° C. Sequencing reactions were primed using the NeoC primer (ATCTTGTTCAATCATGCG (SEQ ID NO. 1)), and fractionated on a Betagen AutoTrans apparatus at 950 constant volts and transferred onto 60 cm of nylon membrane at a 2.0 web speed and a 450 min. web time.

Throughout this application various publications are referenced. Certain publications are referenced by numbers within parentheses. Full citations for the number-referenced publications are listed below. The disclosures of all of these publications and those references cited within those publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the claims included herein.

TABLE 1
Genes disrupted by tagged sequence mutagenesis.

| Functional | | Database | | Score Next | |
|---|---|---|---|---|---|
| Group | Gene | Sequence | Score | Best Match | Gene Function |
| DNA Binding | FBP | gb I U05040 | 1.1e−05* | none | far-upstream binding protein, c-myc gene regulation |
| | NonO | gb I S64860 | 7.4e−14 | none | homologue of the Drosophila nonA^{diss} gene |
| | NACA | gb I U48363 | 2.9e−22 | none | muscle specific transcription factor |
| | Histone H1 | gb I M29260 | 2.8e−26 | none | core nucleosome component |
| RNA Binding | hnRNP F | gb I L28010 | 3.0e−12 | 0.082 | RNA processing, gene regulation |
| | hnRNPA2/B1 | dbj I D28877 | 1.2e−12 | none | RNA processing, gene regulation |
| | polyA BP II | emb I X89969 | 1.8e−12 | none | mRNA polyadenylation subunit |
| | SAP49 | gb I L35013 | 2.2e−20 | none | spliceosome protein |
| | FBRNP | gb S63912 | 1.8e−33 | 0.002 | similar to hnRNPs, expressed in fetal brain |
| | EWS | emb I X72990 | 1.8e−33 | none | translocated in Ewing's sarcoma, fusing with Fli1 & |
| | | | | | other DNA BP |
| | fus/TLS | gb I U36561 | 6.8e−38 | none | translocated in solid tumors, fusing with CHOP & other |
| | | | | | DNA BP |
| | Deadbox | gb I L25125 | 1.9e−62 | 0.0057 | RNA helicase and RNA-dependant ATPase from |
| | | | | | DEAD box vamily |
| Translation | L29 (3) | emb I Z49148 | 2.2e−09 | none | ribosome subunit protein |
| | S19 | emb I X51707 | 5.5e−17 | none | ribosome subunit protein |
| | L27a | emb I X52733 | 1.1e−17 | none | ribosome subunit protein |
| | fau/S30 | gb I L33715 | 2.7e−46 | none | ribosome subunit protein, FBR-MuSV fox sequence |
| | L19 | emb I X82202 | 4.1e−61 | none | ribosome subunit protein |
| Metabolic | AIR-C | dbj I D37978 | 4.1e−32 | none | aminoimidazole ribonucleotide carboxylase' purine |
| Enzymes | | | | | biosynthesis |
| | Asp Syn'tase | gb I U38940 | 9.1e−56 | none | asparagine synthetase |
| | tri-pep II | emb I X81323 | 2.5e−56 | none | tripeptidyl peptidase II, intracellular exopeptidase |
| Cell | Laminin R | gb I M27798 | 2.5e−26 | none | 67 kD high affinity laminin binding protein, induced in |
| Surface/Matrix | | | | | transfonned cells |
| | Filamin‡ | pirIA49551 | 2.1e−31 | 0.27 | endothelial actin-binding protein |
| | α-NAC (2)¶ | gb IU48363 | 3.8e−95 | none | nascent polypeptide-associated complex |
| Signal | fnk | gb I U21392 | 1.4e−20 | none | serine-threonine kinase, basic FGF signalling |
| Transduction | | | | | |
| | GRK6 | gb I L16862 | 4.3e−22 | none | G-protein coupled receptor kinase |
| | plk | gb I L06144 | 1.4e−34 | none | serine-threonine kinase, poio and CDC5 homologue |
| Protein Kinesis | extendin | gb I U27830 | 2.7e−40 | none | cell motility, protein localized to extending psuedopodia |
| Unknown | PM-sc1 | db I U09215 | 5.8e−23 | −.0042 | 75 kD nuclear autoantigen |
| | gas5 | emb I X67267 | 5.7e−42 | none | gene induced in growth arrested cells |
| | Ly-6 linked^{#} | gb I M37707 | 1.1e−77 | none | gene adjacent to Ly-6 stem cell differentiation antigen |
| | GLUT1^{#} | dbj I D10231 | 3.1e−93 | none | erythrocyte glucose transporter |
| Retroposons | IE118 | emb I X13056 | 1.4e−18 | none | mouse insertion element |
| | LINE-1⁺ | gb I S64180 | 3.5e−20 | none | long interspersed element, A-monomer |
| | LINE-1 | emb I X04318 | 1.2e−26 | none | long interspersed element, A-monomer |
| | LINE-1 | emb I X59221 | 2.4e−41 | none | long interspersed element, A-monomer |
| | LINE-1 | emb I X04318 | 6.2e−51 | none | long interspersed element, A-monomer |
| | LINE-1 | emb I X59214 | 2.7e−51 | none | long interspersed element, A-monomer |
| | LINE-1 | gb I S64180 | 1.0e−74 | none | long interspersed element, A-monomer |
| | VL30 | gb I M76549 | 8.le−91 | none | virus-like 30S element |

Table 1. Genes disrupted by tagged sequence mutagenesis. Comparison of 400 PSTs with the non-redundant GenBank database revealed 42 previously characterized genes disrupted as a result of virus integration. Matching genes, database entry of matching gene sequences, and functional information about each gene are listed. Scores represents the probability of the BLASTN match occurring by chance alone. Scores for sequences (if any) producing the next most significant match are also provided to illustrate the relative significance of each gene-PST match. *Probability scores of less than 10e-8 were considered significant. In addition to the criteria outlined in the text; the match with the least significant score, involving FBP, was confirmed by the identification of another exon in the flanking DNA (data not shown). # All proviruses were in or near 5′ exons of the identified genes except (i) GLUT1, which inserted 4.2 Kb into the second intron possibly identifying an alternative promoter for GLUT1 transcripts is active in ES cells, or (ii) Provirus insertion 2.7 Kb upstream of the 5′ end of the Ly-6E gene and in the opposite transcriptional orientation, suggesting the existence of cellular gene positioned head to head with respect to Ly-6E. ‡Filamin was first scored as an EST match which identified exons within the PST. The score and identification of filamin presented here is from a protein search of the predicted amino acid translation of the PST. ¶α-NAC was independently targeted three times. The third defined mutation occurred in an alternatively spliced exon which has been shown to convert the molecule to a transcription factor, NACA. ⁺All Line-1 inserts occurred in 5′ A-monomer repeat regions present only in full-length elements. Moreover, at least one intact A-monomer was upstream of all inserts, consistent with the presence of a functional promoter in the repeat.

TABLE 2
ESTs disrupted by tagged sequence mutagenesis.

| Cell Line/PST | Matching EST | Acces. No. | Species | Score |
|---|---|---|---|---|
| E21C | II9638.seq | gbAA092816 | mouse | 0.0015 |
| H7E | mj99g08.r1 | gbA080237 | mouse | 1.1e−06 |
| H2B | C06719 | dbjC067l9 | rat | 9.0e−07 |
| HK18 | mp53e11.r1 | gbAA111690 | mouse | 1.3e12 |
| E22H | zn63d11.r1 | gbAA1006l4 | human | 1.3e−12 |
| H19K | mm33d10.r1 | gbAA079906 | mouse | 2.5e−12 |
| HE48G | mi63c12.r1 | gbAA014252 | mouse | 1.4e−14 |
| E1K | E5T112024 | gbH34788 | rat | 1.4e−14 |
| HO22 | sap27k | embX94514 | mouse | 2.6e−16 |
| HN11 | KIAA0259 | dbjD87077 | human | 1.9e−17 |
| H17D | mb60e04.r1 | gbW16061 | mouse | 1.4e−17 |
| HE15Q | mm87d08.r1 | gbAA087300 | mouse | 9.7e−19 |
| HE14R | CMG5 | gbM83344 | mouse | 9.1e−23 |
| E2F | mo08d03.r1 | gbAA097618 | mosue | 4.5e−28 |
| E14A | D86678 | dbjD86678 | rat | 2.6e−30 |
| H7C-1 | mj43d09.r1 | gbAA048831 | mouse | 2.0e−38 |
| E23G | mo15a03.r1 | gbAA097108 | mouse | 3.6e−46 |
| HM17 | KIAA0240 | dbjDS7077 | human | 2.1e−53 |
| H24D | yv88h07.r1 | gbH85526 | human | 2.5e−57 |
| E5L | mm87d08.r1 | gbAA087300 | mouse | 2.5e−72 |
| HM16 | mb94d05.r1 | gbW36740 | mosue | 2.8e−80 |

Table 2. ESTs disrupted by tagged sequence mutagenesis. Comparison of 400 PSTs with the GenBank EST database (DBEST) revealed 21 inserts into genes previously characterized as anonymous cDNAs (ESTs) which were not identified in Table 1. Cell lines from which the PSTs were derived are listed together with names, accession numbers, and species of origin of matching ESTs and the score of the EST-PST match.

References

1. Gibbs, R. A. Pressing ahead with human genome sequencing. Nature Genet. 11, 121-125 (1995).

2. Oliver, S. G. From DNA sequence to biological function. Nature 379, 597-600 (1996).

3. McKusick, V. A. Mendelian Inheritance in Man: Catalogue of Autosoma Dominant, Autosomal Recessive, and X-Linked Phenotypes, 1626 (The John Hopkins Univ. Press, Baltimore, 1988).

4. Green, M. C. Catalog of mutant genes and polymorphic loci. in Genetic variants and strains of the laboratory mouse. (eds Lyon, M. F. & Searle, A. G.) 12-403. (Oxford University Press, Oxford, U.K., 1989).

5. Reith, A. D. & Berstein, A. Molecular basis of mouse developmental mutants. Genes Devel. 5, 1115-1123 (1991).

6. Capecchi, M. R. Altering the genome by homologous recombination. Science 244, 1288-1292 (1989).

7. Brandon, E. P., Idzerda, R. L. & S., M. G. Targeting the mouse genome: a compendium of knockouts (part I). Current Biology 5, 625-634 (1995).

8. Gossler, A., Joyner, A. L., Rossant, J. & Skarnes, W. C. Mouse embryonic stem cells and reporter constructs to detect developmentally regulated genes. Science 244, 463-465 (1989).

9. Friedrich, G. & Soriano, P. Promoter traps in embryonic stem cells: a genetic screen to identify and mutate developmental genes in mice. Genes Dev. 5, 1513-1523 (1991).

10. von Melchner, H. et al. Selective disruption of genes expressed in totipotent embryonal stem cells. Genes Dev. 6, 919-927 (1992).

11. Skarnes, W. C., Auerbach, B. A. & Joyner, A. A gene trap approach in mouse embryonic stem cells: the lacZ reporter is activated by splicing reflects endogenous gene expression, and is mutagenic in mice. Genes Dev. 6, 903-918 (1992).

12. Skarnes, W. C., Moss, J. E., Hurtley, S. M. & Beddington, R. S. Capturing genes encoding membrane and secreted proteins important for mouse development.

Proc Natl Acad Sci U S A 92, 6592-6 (1995).

13. Scherer, C. A., Chen, J., Nachabeh, A., Hopkins, N. & Ruley, H. E.

Transcriptional specificity of the pluripotent embryonic stem cell. Cell: Growth Diff 7, 1393-1401 (1996).

14. Forrester, L. M. et al. An induction gene trap screen in embryonic stem cells: Identification of genes that respond to retinoic acid in vitro. Proc. Natl. Acad.

Sci. 93, 1677-1682 (1996).

15. Reddy, S., Rayburn, H., von Melchner, H. & Ruley, H. E. Fluorescence-activated sorting of totipotent embryonic stem cells expressing developmentally regulated lacZ fusion genes. Proc. Natl. Acad. Sci. USA 89, 6721-6725 (1992).

16. Wurst, W. et al. A large-scale gene-trap screen for insertional mutations in developmentally regulated genes in mice. Genetics 139, 889-899 (1995).

17. Nussbaum, R. L., Lesko, J. G., Lewis, R. A., Ledbetter, S. A. & Ledbetter, D. H. Isolation of anonymous DNA sequences from within a submicroscopic X chromosomal deletion in a patient with choroideremia, deafness, and mental retardation. Proc. Natl. Acad. Sci. USA 84, 6521-6525 (1987).

18. Chang, W., Hubbard, C., Friedel, C. & Ruley, H. E. Enrichment of insertional mutants following retrovirus gene trap selection. Virology 193, 737-747 (1993).

19. Withers-Ward, E. S., Kitamura, Y., Barnes, J. P. & Coffin, J. M. Distribution of targets for avian retrovirus DNA integration in vivo. Genes Dev. 8, 1473-1487 (1994).

20. Altschul, S. F., Gish, W., Miller, W., Meyers, E. W. & Lipman, D. J. Basic local alignment search too. J. Mol. Biol. 215, 403-410 (1990).

21. Adams, M. D. et al. Sequence identification of 2,375 human brain genes. Nature 13, 632-634 (1992).

22. Adams, M. D. et al. Complementary DNA sequencing: expressed sequence tags and human genome project. Science 252, 1643-1651 (1991).

23. Adams, M. D., Kerlavage, A. R., Fields, C. & Venter, J. C. 3,400 new expressed sequence tags identify diversity of transcripts in human brain. Nat Genet 4, 256-267 (1993).

24. Okubo, K. et al. Large scale cDNA sequencing for analysis of quantitative and qualitative aspects of gene expression. Nature Genet. 2, 180-185 (1992).

25. Nehls, M., Pfeifer, D., Micklem, G., Schmoor, C. & Boehm, T. The sequence complexity of exons trapped from the mouse genome. Curr. Biology 4, 983-989 (1994).

26. Crozat, A., Aman, P., Mandahl, N. & Ron, D. Fusion of CHOP to a novel RNA-binding protein in human myxoid liposarcoma. Nature 363, 640-644 (1993).

27. Rabbitts, T. H., Forster, A., Larson, R. & Nathan, P. Fusion of the dominant negative transcription regulator CHOP with a novel gene FUS by translocation t (12;16) in malignant liposarcoma. Nature Genet. 4, 175-180 (1993).

28. Zucman, J. et al. Combinatorial generation of variable fusion proteins in the Ewing family of tumours. Embo J 12, 4481-4487 (1993).

29. Zucman, J. et al. EWS and ATF-1 gene fusion induced by t(12;22) translocation in malignant melanoma of soft parts. Nat Genet 4, 341-345 (1993).

30. Llamazares, S. et al. polo encodes a protein kinase homolog required for mitosis in Drosophila. Genes Dev 5, 2153-2165 (1991).

31. Clay, F. J., McEwen, S., Bertoncello, I., Wilks, A. F. & Dunn, A. R. Identification and cloning of a protein kinase encoding mouse gene Plk, related to the polo gene of Drosophila. Proc. Natl. Acad. Sci. U.S.A. 90, 4882-4886 (1993).

32. Fenton, B. & Glover, D. M. A conserved mitotic kinase active at late anaphase-telophase in syncytial Drosophila embryos. Nature 363, 637-640 (1993).

33. Lake, R. J. & Jelinek, W. R. Cell cycle- and terminal differentiation-associated regulation of the mouse mRNA encoding a conserved mitotic protein kinase. Mol. Cell. Biol. 13, 7793-7801 (1993).

34. Rendahl, K. G., Jones, K. R., Kulkarni, S. J., Bagully, S. H. & Hall, J. C. The dissonance mutation at the no-on-transient-A locus of D. melanogaster: genetic control of courtship song and visual behaviors by a protein with putative RNA-binding motifs. J Neurosci 12, 390-407 (1992).

35. Yang, Y. S. et al. NonO, a non-POU-domain-containing, octamer-binding protein, is the mammalian homolog of Drosophila nonAdiss. Mol. Cell. Biol. 13, 5593-5603 (1993).

36. Duncan, R. et al. A sequence-specific, single-strand binding protein activates the far upstream element of c-myc and defines a new DNA-binding motif Genes Dev 8, 465-480 (1994).

37. Coccia, E. M. et al. Regulation and expression of a growth arrest-specific gene (gas5) during growth, differentiation, and development. Mol. Cell. Biol. 12, 3514-3521 (1992).

38. DeGregori, J. et al. A murine homolog of the yeast RNA1 gene is required for postimplantation development. Genes Dev. 8, 265-276 (1993).

39. Chen, J. et al. Germline inactivation of the murine eck receptor tyrosine kinase by gene trap retroviral insertion. Oncogene 12, 979-988 (1996).

40. Lewin, B. Units of transcription and translation: sequence components of heterogeneous nuclear RNA and messenger RNA. Cell 4, 77-93 (1975).

41. Chen, Z., Friedrich, G. A. & Soriano, P. Transcriptional enhancer factor 1 disruption by a retroviral gene trap leads to heart defects and embryonic lethality in mice. Genes Dev 8, 2293-2301 (1994).

42. Deng, J. M. & Behringer, R. R. An insertional mutation in the BTF3 transcription factor gene leads to an early postimplantation lethality in mice. Transgenic Res 4, 264-269 (1995).

43. Gasca, S., Hill, D. P., Klingensmith, J. & Rossant, J. Characterization of a gene trap insertion into a novel gene, cordon-bleu, expressed in axial structures of the gastrulating mouse embryo. Dev Genet 17, 141-154 (1995).

44. Takeuchi, T. et al. Gene trap capture of a novel mouse gene, jumonji, required for neural tube formation. Genes Dev 9, 1211-1222 (1995).

45. Hutchison III, C. A., Hardies, S. C., Loeb, D. D., Shehee, W. R. & Edgell, M. H. Lines and related retroposons: long interspersed repeated sequences in the eucaryotic genome. in Mobile DNA (eds Berg, D. E. & Howe, M. M.) 593-617 (Am. Soc. Microbiol., Washington, D.C., 1989).

46. Shih, C. C., Stoye, J. P. & Coffin, J. M. Highly preferred targets for retrovirus integration. Cell 53, 531-537 (1988).

47. Sandmeyer, S. B., Hansen, L. J. & Chalker, D. L. Integration specificity of retrotransposons and retroviruses. Ann. Rev. Genet. 24, 491-518 (1990).

48. Monk, M., Boubelik, M. & Lehnert, S. Temporal and regional changes in DNA methylation in the embryonic, extraembryonic and germ cell lineages during mouse embryo development. Development 99, 371-382 (1987).

49. Kafri, T. et al. Developmental pattern of gene-specific DNA methylation in the mouse embryo and germ line. Genes Dev. 6, 705-714 (1992).

50. Lowe, S. W., Ruley, H. E., Jacks, T. & Housman, D. E. p53-dependent apoptosis modulates the cytotoxicity of anticancer agents. Cell 74, 957-967 (1993).

51. Lowe, S. W., Schmitt, E. M., Smith, S. W., Osborne, B. A. & Jacks, T. p53 is required for radiation-induced apoptosis in mouse thymocytes. Nature 362, 847-849 (1993).

52. Kastan, M. B. et al. A mammalian cell cycle checkpoint pathway utilizing p53 and GADD45 is defective in ataxia-telangiectasia. Cell 71, 587-597 (1992).

53. Mortensen, R. M., Conner, D. A., Chao, S., Geisterfer-Lowrance, A. A. & Seidman, J. G. Production of homozygous mutant ES cells with a single targeting construct. Mol. Cell. Biol. 12, 2391-2395 (1992).

54. Yotov, W. V. & St-Arnaud, R. Differential splicing-in of a proline-rich exon converts alphaNAC into a muscle-specific transcription factor. Genes Dev. 10, 1763-1772 (1996).

55. Ramirez-Solis, R., Liu, P. & Bradley, A. Chromosome engineering in mice. Nature 378, 720-724 (1995).

56. Chen, J. et al. Retrovirus Gene Traps. Meth Mol Genet 4, 123-140 (1994).

57. Hicks, G. G. et al. Retrovirus Gene Traps. Methods Enzymol 254, 263-275 (1995).

58. Hsiao, K. A fast and simple procedure for sequencing double stranded DNA with Sequenase. Nucleic Acids Res. 19, 2787. (1991).

What is claimed is:

1. A method of producing a selected cell line or a non-human transgenic animal model for the analysis of the function of a gene comprising:
a) introducing into an embryonic stem cell a vector having a selectable marker which, when the vector is inserted within a gene, the inserted vector can inhibit the expression of the gene;
b) selecting embryonic stem cells expressing the selectable marker;
c) excising the vector from the embryonic stem cells expressing the selectable marker such that host DNA from the gene is linked to the excised vector;
d) sequencing the host DNA in the excised vector;
e) comparing the sequence of the host DNA to known gene sequences to determine which host DNA is from a gene for which a model for the analysis of the function the gene is desired;
f) selecting the embryonic stem cell containing the inhibited gene for which a model for the analysis of gene function is desired; and
g) forming a cell line or a non-human transgenic animal from the selected embryonic stem cell.

2. The method of claim 1, wherein step (f) further comprises isolating the embryonic stem cell containing the inhibited gene for which a model for the analysis of gene function is desired from the other embryonic stem cells expressing the selectable marker.

3. The method of claim 1, wherein the vector comprises an origin of replication specific to a replication host and wherein the vector is excised from the embryonic stem cell and introduced into the replication host cell prior to sequencing.

4. The method of claim 1, wherein in step (f) the inhibited gene is a previously unidentified gene.

5. The method of claim 3, wherein the replication host is a prokaryote.

6. The method of claim 3, wherein the replication host is a yeast.

7. The method of claim 5, wherein the origin of replication is prokaryotic and the replication host is E. coli.

8. The method of claim 1, wherein the embryonic stem cell is a murine embryonic stem cell.

9. The method of claim 1, wherein the vector is a retroviral vector.

10. The method of claim 9, wherein the vector comprises a defective Moloney leukemia virus.

11. The method of claim 9, wherein the vector further comprises a non-mammalian origin of replication.

12. The method of claim 9, wherein the origin of replication is prokaryotic.

13. The method of claim 9, wherein the origin of replication is from yeast.

14. The method of claim 9, wherein the vector comprises U3NeoSV1.

15. The method of claim 9, wherein the vector further comprises an enhancement sequence that can be used to enhance the isolation of the retroviral vector.

16. The method of claim 15, wherein the enhancement sequence comprises the lac operator.

17. A library of embryonic stem cells produced by the method of claim 1.

18. A transgenic animal produced by the method of claim 1.

19. A cell line produced by the method of claim 1.

20. A replication host cell containing a vector produced by the method of claim 3.

21. Replication host cells produced by the method of claim 3.

22. A library of embryonic stem cells wherein 1) a multiplicity of cells in the library each contain a gene having inhibited expression, 2) a sequence of the gene having inhibited expression is known, and 3) a multiplicity of different non-functional genes is represented in the library.

23. The library of embryonic stem cells of claim 22, wherein the cells further comprise a retroviral shuttle vector having a selectable marker and an origin of replication specific to a replication host.

24. The shuttle vectors of claim 23, wherein the origin of replication is non-mammalian.

25. A method of selecting a cell line or a non-human transgenic animal model for the analysis of the function a gene comprising:
a) introducing into an embryonic stem cell a vector having a selectable marker which, when the vector is inserted within the gene, the inserted vector can inhibit the expression of the gene;
b) selecting embryonic stem cells expressing the selectable marker;
c) excising the vector from the embryonic stem cells expressing the selectable marker whereby host DNA from the gene is linked to the excised vector;
d) sequencing host DNA in the excised vector;
e) comparing the sequence of the host DNA to known gene sequences to determine which host DNA is from a gene for which a model for the analysis of the function the gene is desired; and
f) selecting the embryonic stem cell containing the inhibited gene for which a model for the analysis of gene function is desired.

26. A method of creating a library of embryonic stem cells wherein 1) a multiplicity of cells in the library each contain a gene having inhibited expression, 2) a sequence of the gene having inhibited expression is known, and 3) a multiplicity of different inhibited genes is represented in the library, comprising
a) introducing into an embryonic stem cell a vector having a selectable marker which, when the vector is inserted within a gene, the inserted vector can inhibit the expression of the gene;
b) selecting embryonic stem cells expressing the selectable marker;
c) excising the vector from the embryonic stem cells expressing the selectable marker such that host DNA from the gene is linked to the excised vector; and
d) sequencing the host DNA in the excised vector; thereby identifying sequence of the gene whose expression is inhibited, and creating a library of embryonic stem cells containing the gene whose expression is inhibited and a sequence of the inhibited gene is known.

27. The method of claim 26, further comprising isolating the embryonic stem cell containing the inhibited gene from the other embryonic stem cells expressing the selectable marker.


SEQUENCE LISTING (1 sequence)

SEQ ID NO: 1 - DNA (oligonucleotide), 18 bp; single-stranded, linear
atcttgttcaatcatgcg